(19) United States
(12) Patent Application Publication
(10) Pub. No.: US 2023/0356713 A1
(43) Pub. Date: Nov. 9, 2023

(54) Driver Assessment System to Determine a Driver's Ability to Perform Driving Tasks

(71) Applicant: Robert Bosch GmbH, Stuttgart (DE)

(72) Inventor: Michael J. Bowyer, Livonia, MI (US)

(21) Appl. No.: 17/739,650

(22) Filed: May 9, 2022

(51) Int. Cl. B60W 30/09; G06N 20/00; A61B 5/18; A61B 5/00

(52) U.S. Cl. CPC B60W 30/09; G06N 20/00; A61B 5/18; A61B 5/681; A61B 5/7267; B60W 2540/221; B60W 2540/30; B60W 2556/45

(57) ABSTRACT

Systems and devices for determining a current capability of a driver of a vehicle. One embodiment provides an electronic processor to receive, from a smart device associated with a driver of the vehicle, driver data that includes an indicator regarding an ability of the driver to drive the vehicle; process the driver data through a first model to determine a current capability of the driver, the first model trained with previously received driver data for the driver; determine, based on the current capability of the driver and a current state of the vehicle, a command to control or prevent control of the vehicle; and provide the command to a system of the vehicle for execution.

### BACKGROUND

Many car accidents occur due to driver's physical or mental condition, and especially due to the driver's stress, tiredness, or due to other medical problem.

### SUMMARY OF THE DESCRIPTION

The present disclosure provides for driver assessment systems that determine the ability of a driver of a vehicle to perform a driving task and, in some instances, disables a function or controls the vehicle to perform a maneuver to help reduce the likelihood of a collision or other undesirable event involving the vehicle.

In one aspect, disclosed herein, are systems for determining a current capability of a driver of a vehicle that include a processor. The processor is configured to receive, from a smart device associated with a driver of the vehicle, driver data that includes an indicator regarding an ability of the driver to drive the vehicle; process the driver data through a first model to determine a current capability of the driver, the first model trained with previously received driver data for the driver; determine, based on the current capability of the driver and a current state of the vehicle, a command to control or prevent control of the vehicle; and provide the command to a system of the vehicle for execution. In some examples, the first model is retrained with the determined current capability of the driver. In some examples, the first model is retrained with the driver data. In some examples, the first model trained with previously received driver data for other drivers of the vehicle or other vehicles. In some examples, the command is determined by processing the current capability of the driver and the current state of the vehicle through a second model. In some examples, the second model is trained with previously received driver data for the driver, previously determined capability of the other drivers or respective vehicle states for the other vehicles. In some examples, the second model is retrained with the determined command. In some examples, the second model is retrained with the current state of the vehicle. In some examples, the current capability of the driver includes a measure of the ability of the driver to drive the vehicle. In some examples, the ability of the driver to drive the vehicle includes the driver's cognitive ability to perform at least one dynamic driving task. In some examples, the driver data includes key indicators of the driver's ability to recognize and react to danger. In some examples, the key indicators include biometric data, driving behavior data, and health related data. In some examples, the system includes an Advanced Driver Assistance System (ADAS). In some examples, the current state of the vehicle is received from the ADAS. In some examples, the ADAS is executed by an electronic control unit (ECU) of the vehicle. In some examples, the smart device includes an item of smart jewelry worn by the driver. In some examples, the smart device includes a mobile device coupled to a smart watch worn by the user. In some examples, the biometric data is collected by the smart watch that is provided to the mobile device. In some examples, the command includes disabling a function of the vehicle or performing a minimum risk maneuver. In some examples, the processor is housed within a mobile device. In some examples, the processor is a component of an ECU of the vehicle. In some examples, the processor is housed within the smart device. In some examples, the smart device is communicably coupled to the vehicle via an infotainment system of the vehicle.

In another aspect, disclosed herein, are methods for determining a current capability of a driver of a vehicle. These methods include receiving, from a smart device associated with a driver of the vehicle, driver data that includes an indicator regarding an ability of the driver to drive the vehicle; processing the driver data through a first model to determine a current capability of the driver, the first model trained with previously received driver data for the driver; determining, based on the current capability of the driver and a current state of the vehicle, a command to control or prevent control of the vehicle; and providing the command to a system of the vehicle for execution. In some examples, the first model is retrained with the determined current capability of the driver. In some examples, the first model is retrained with the driver data. In some examples, the first model trained with previously received driver data for other drivers of the vehicle or other vehicles. In some examples, the command is determined by processing the current capability of the driver and the current state of the vehicle through a second model. In some examples, the second model is trained with previously received driver data for the driver, previously determined capability of the other drivers or respective vehicle states for the other vehicles. In some examples, the second model is retrained with the determined command. In some examples, the second model is retrained with the current state of the vehicle. In some examples, the current capability of the driver includes a measure of the ability of the driver to drive the vehicle. In some examples, the ability of the driver to drive the vehicle includes the driver's cognitive ability to perform at least one dynamic driving task. In some examples, the driver data includes key indicators of the driver's ability to recognize and react to danger. In some examples, the key indicators include biometric data, driving behavior data, and health related data. In some examples, the system includes an ADAS. In some examples, the current state of the vehicle is received from the ADAS. In some examples, the ADAS is executed by an ECU of the vehicle. In some examples, the smart device includes an item of smart jewelry worn by the driver. In some examples, the smart device includes a mobile device coupled to a smart watch worn by the user. In some examples, the biometric data is collected by the smart watch that is provided to the mobile device. In some examples, the command includes disabling a function of the vehicle or performing a minimum risk maneuver. In some examples, the methods are executed by a processor. In some examples, the processor is housed within a mobile device. In some examples, the processor is a component of an ECU of the vehicle. In some examples, the processor is housed within the smart device. In some examples, the smart device is communicably coupled to the vehicle via an infotainment system of the vehicle.

In another aspect, disclosed herein, are non-transitory computer-readable medium that include instructions executable by an electronic processor to perform a set of functions. This set of functions includes receiving, from a smart device associated with a driver of the vehicle, driver data that includes an indicator regarding an ability of the driver to drive the vehicle; processing the driver data through a first model to determine a current capability of the driver, the first model trained with previously received driver data for the driver; determining, based on the current capability of the driver and a current state of the vehicle, a command to control or prevent control of the vehicle; and providing the command to a system of the vehicle for execution. In some examples, the first model is retrained with the determined current capability of the driver. In some examples, the first model is retrained with the driver data. In some examples, the first model trained with previously received driver data for other drivers of the vehicle or other vehicles. In some examples, the command is determined by processing the current capability of the driver and the current state of the vehicle through a second model. In some examples, the second model is trained with previously received driver data for the driver, previously determined capability of the other drivers or respective vehicle states for the other vehicles. In some examples, the second model is retrained with the determined command. In some examples, the second model is retrained with the current state of the vehicle. In some examples, the current capability of the driver includes a measure of the ability of the driver to drive the vehicle. In some examples, the ability of the driver to drive the vehicle includes the driver's cognitive ability to perform at least one dynamic driving task. In some examples, the driver data includes key indicators of the driver's ability to recognize and react to danger. In some examples, the key indicators include biometric data, driving behavior data, and health related data. In some examples, the system includes an ADAS. In some examples, the current state of the vehicle is received from the ADAS. In some examples, the ADAS is executed by ECU of the vehicle. In some examples, the smart device includes an item of smart jewelry worn by the driver. In some examples, the smart device includes a mobile device coupled to a smart watch worn by the user. In some examples, the biometric data is collected by the smart watch that is provided to the mobile device. In some examples, the command includes disabling a function of the vehicle or performing a minimum risk maneuver. In some examples, the processor is housed within a mobile device. In some examples, the processor is a component of an ECU of the vehicle. In some examples, the processor is housed within the smart device. In some examples, the smart device is communicably coupled to the vehicle via an infotainment system of the vehicle.

It is appreciated that methods in accordance with the present disclosure can include any combination of the aspects and features described herein. That is, methods in accordance with the present disclosure are not limited to the combinations of aspects and features specifically described herein, but also may include any combination of the aspects and features provided.

The details of one or more embodiments of the present disclosure are set forth in the accompanying drawings and the description below. Other features and advantages of the present disclosure will be apparent from the description and drawings, and from the claims.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate examples of concepts that include the claimed invention and explain various principles and advantages of those examples.

FIG. 1 depicts an example environment according to some aspects.

FIG. 2 depicts an example architecture according to some aspects.

FIG. 3 depicts a flowchart of an example process according to some aspects.

FIG. 4 depicts a block diagram of an example system that includes a computing device that can be programmed or otherwise configured according to some aspects.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of examples and aspects described and illustrated.

The apparatus and method components have been represented where appropriate by conventional symbols in the drawings, showing only those specific details that are pertinent to understanding the examples and aspects disclosed so as not to obscure the disclosure with details that will be readily apparent to those of ordinary skill in the art having the benefit of the description herein.

### DETAILED DESCRIPTION

Generally, one example driver assessment system determines the ability of a driver of a vehicle to perform a driving task and, in some instances, disables a function or controls the vehicle to perform a maneuver to help reduce the likelihood of a collision or other undesirable event involving the vehicle. The ability of a driver of a vehicle to perform a driving task may include, for example, whether the driver can recognize danger, react to danger, and react in time to danger. For example, the system may determine whether a driver is awake, conscious, or has the cognitive ability to drive the vehicle. In some cases, the current capability of the driver is determined by processing information related to the driver (driver data) that includes key indicators of the driver's ability to drive. The key indicators include, for example, biometric data (e.g., heart rate, blood oxygen levels), driving behavior data (e.g., steering behavior data), and health related data (e.g., quality and amount of sleep received, caloric intake, recent exercise intensity, and so forth) and are provided to the driver assessment system by various onboard sensors or sensors (e.g., a smart device) wore by the driver. For example, a driver may enter the driver data into their smart device(s), or the driver data may be collected by the smart device(s) automatically. Given these key indicators of drivers' ability to recognize and react to danger the system prevents function activations as well as fall back to driver as safety mechanism when driver is unable to perform full driving task (e.g., stay in lane, travel at safe speed, prevent collisions, and so forth).

In some examples, the driver assessment system monitors the interior of the respective vehicle. The system detects, for example, driver distraction, signs of drowsiness, and whether a child has been left behind in the vehicle and may provide alerts to the driver in predetermined situations. In some cases, safety systems, for example, the seat belt alert function, are enhanced using information provided by the described system.

In some examples, the driver assessment system processes received driver information through a trained driving ability model to determine a current capability of the driver. In one example, the driver assessment system receives, from a steering-angle sensor or a smart device worn by the driver, driver information that includes biometric data for the driver or data related to the driver's steering or use of turn signals. The driver information along with other relevant information, for example, the length of the current trip, the time of day, and so forth, is processed through the trained driving ability model to determine a capability of the driver, which, for example, includes the driver's level of fatigue. In some instances, the system continuously processes collected driver information during a trip through the driving ability model to determine and update the capability of the driver.

In some cases, the capability of the driver along with a current state of the vehicle is processed through a trained command generation module to determine a command for an ADAS or an infotainment unit. In some cases, a determined command includes displaying information to the driver via, for example, and infotainment unit. For example, the command may display a coffee cup icon on an instrument panel to warn drivers that they need to rest. In some cases, the drivers' smart device is coupled to the infotainment unit and the information is provided to the smart device for display or to initiate an alert.

In some instances, the driver assessment system retrains the driving ability model and the command generation module with the determined capability of the driver and commands, respectively. In some case, the models are received and updated from a back-end system via connected network. In some cases, the system provides the determined capability of the driver and commands to the back-end system for retraining of the respective models. In some instances, the system generates a driver's profile for each unique individual who drives the respective vehicle. In some cases, a driver's profile is processes through the driving ability model or the command generation module, along with the driver information or determine capability of the driver, to determine the respective output.

In some instances, the driver assessment system is employed to improve a driver's understanding and decision making in terms of function availability and driver hand off events. In some cases, the driver assessment system is employed to improve the performance of ADASs.

One example provides a system for determining a current capability of a driver of a vehicle that includes a processor. The processor is configured to receive, from a smart device associated with a driver of the vehicle, driver data that includes an indicator regarding an ability of the driver to drive the vehicle; process the driver data through a first model to determine a current capability of the driver, the first model trained with previously received driver data for the driver; determine, based on the current capability of the driver and a current state of the vehicle, a command to control or prevent control of the vehicle; and provide the command to a system of the vehicle for execution.

Another example provides a method for determining a current capability of a driver of a vehicle. These method includes receiving, from a smart device associated with a driver of the vehicle, driver data that includes an indicator regarding an ability of the driver to drive the vehicle; processing the driver data through a first model to determine a current capability of the driver, the first model trained with previously received driver data for the driver; determining, based on the current capability of the driver and a current state of the vehicle, a command to control or prevent control of the vehicle; and providing the command to a system of the vehicle for execution.

Another example provides a non-transitory computer-readable medium including instructions executable by an electronic processor to perform a set of functions that include receiving, from a smart device associated with a driver of the vehicle, driver data that includes an indicator regarding an ability of the driver to drive the vehicle; processing the driver data through a first model to determine a current capability of the driver, the first model trained with previously received driver data for the driver; determining, based on the current capability of the driver and a current state of the vehicle, a command to control or prevent control of the vehicle; and providing the command to a system of the vehicle for execution.

### Definitions

Unless otherwise defined, all technical terms used herein have the same meaning as commonly understood by one of ordinary skill in the art to which the present subject matter belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the term “real-time” refers to transmitting or processing data without intentional delay given the processing limitations of a system, the time required to accurately obtain data and images, and the rate of change of the data and images.

### Example Environment

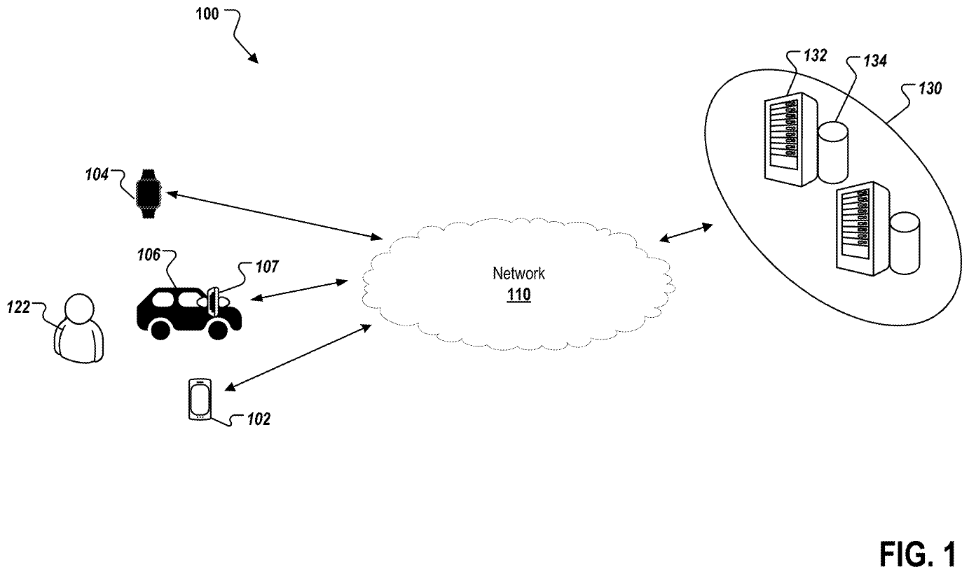

FIG. 1 depicts an example driver assessment system 100. The example system 100 includes computing devices 102 and 104, a vehicle 106, a back-end system 130, and a communication network 110. The vehicle 106 includes various systems, for example, the ADAS or infotainment unit described above that are executed or otherwise communicably coupled to at least one computing device 107 (e.g., via wired or via wireless connections, for example a Bluetooth™ connection). The computing devices 102 and 104 are associated with the driver 122 and are communicably coupled with the various systems that are executed on the computing device 107. For example, the computing devices 102 and 104 may be directly coupled with the computing device 107 via a wireless connection or indirectly via the communications network 110. In some instances, the computing device 107 is an ECU of the vehicle 106.

The communication network 110 may include wireless and wired portions. In some instances, the communication network 110 is implemented using one or more existing networks, for example, a cellular network, the Internet, a near-field communication network (for example, a Bluetooth™ network), a Machine-to-machine (M2M) network, or a public switched telephone network. The communication network 110 may also include future developed networks.

In some instances, the communication network 110 is configured to be accessed over a wired or a wireless communications link. For example, mobile computing devices (e.g., the smartphone device 102, the smart watch 104, the computing device 107) can use a cellular network to access the communications network 110.

In some examples, the driver 122 interacts with the system through a graphical user interface (GUI) or application that is installed and executing on the computing devices 102 and 104 or an infotainment unit installed coupled to the computing device 107. In some examples, the computing devices 102, 104, and 107, provide viewing data to screens with which the driver 122 can interact.

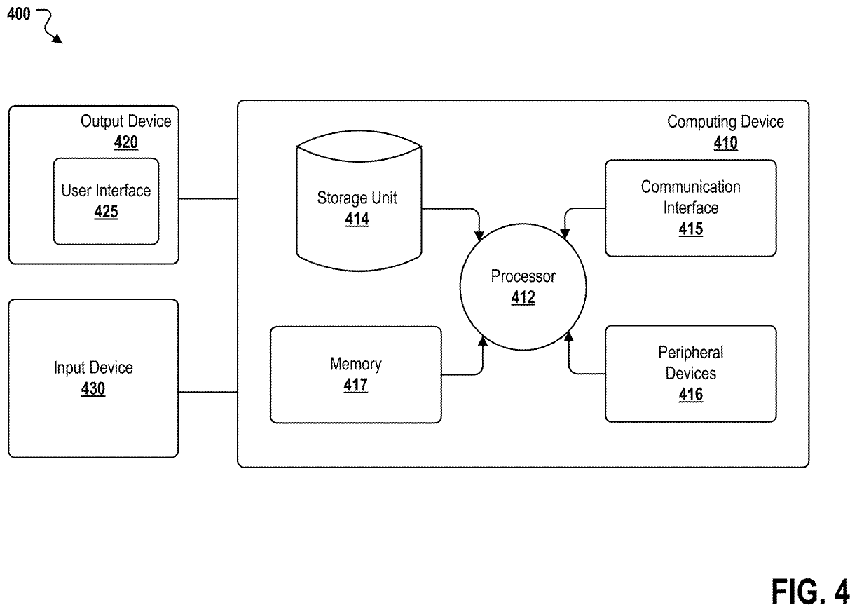

In some instance, the computing devices 102, 104, and 107 are sustainably similar to computing device 410 depicted in FIG. 4.

Three computing devices are depicted in FIG. 1 for simplicity. However, in other instances more or fewer devices may be utilized. In the depicted example environment 100, the computing device 102 is depicted as a smartphone and the computing device 104 is depicted a smart watch. It is contemplated, however, that other computing devices, for example, smart phones, tablet computers, smart jewelry (e.g., fitness rings, fitness anklets) and the like may be utilized.

In some instances, the back-end system 130 includes at least one server device 132 and at least one data store 134. In some aspects, the server device 132 is sustainably similar to computing device 410 depicted in FIG. 4. In some aspects, the back-end system 130 may include server-class hardware type devices. In some aspects, the server device 132 is a server-class hardware type device. In some aspects, the back-end system 130 includes computer systems using clustered computers and components to act as a single pool of resources when accessed through the communications network 110. Such examples may be used in data centers, cloud computing, storage area network (SAN), and network attached storage (NAS) applications. In some aspects, the back-end system 130 is deployed using a virtual machine(s).

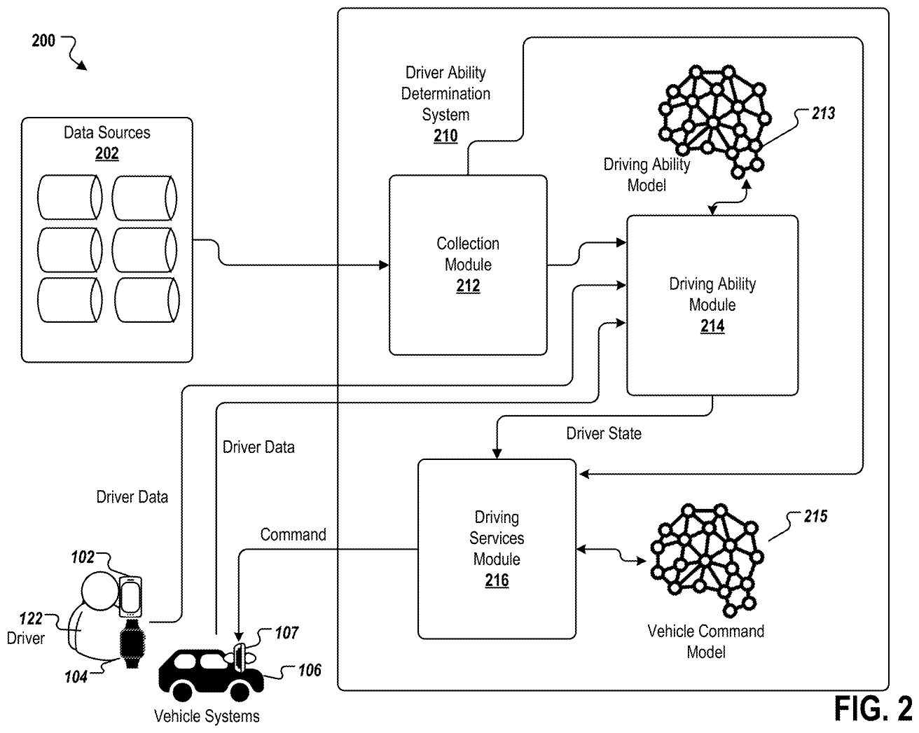

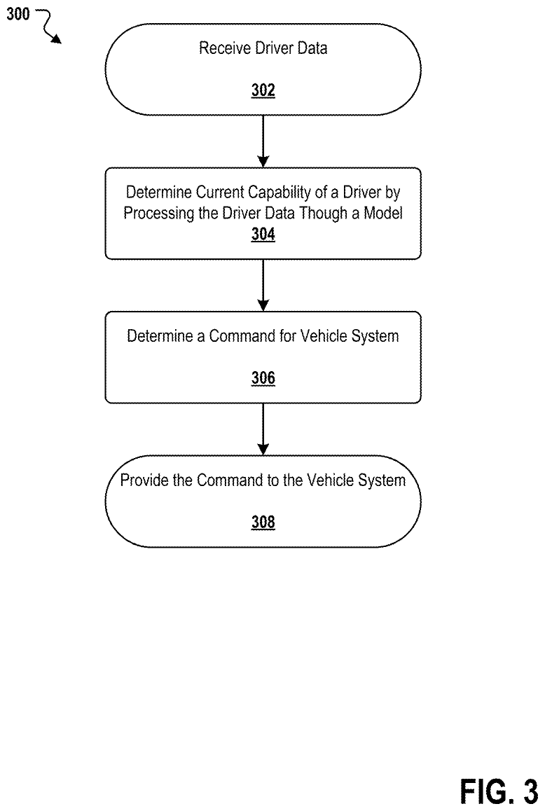

In some examples, the back-end system 130 is employed to train various algorithms (e.g., via machine learning), which are provided to the various systems executing by devices installed on the vehicle 106. For example, the back-end system 130 can be employed to execute the process 300 described with reference to FIG. 3 via the example architecture 200 described with reference to FIG. 2. In other examples, some or all of the components and modules described with reference to FIG. 2 are executed directly by computing device 107.

In some examples, the server system 132 hosts one or more computer-implemented services provided by driver assessment system with which the various systems and devices installed on the computing device 107 can interact via the communications network 110. For example, a system executing on the computing device 107 may receive trained models (e.g., the driving ability model and the command generation module), which are employed to determine a capability of the driver and various commands based on the determined capability of the driver. The system may then provide the determined capability of the driver and commands to the back-end system 130 via the one or more computer-implemented services hosted by the server system 132. In some instances, the models are retrained by modules executing on the server system 132 using the received information. In some cases, these retained models are provided to systems (e.g., the systems executing on the computing device 107) that are connected to the server system 132.

### Example Architecture

FIG. 2 depicts an example architecture 200 for the driver assessment system. The example architecture 200 includes the computing devices 102, 104, and 107 described in FIG. 1; data sources 202, and a driver assessment system 210. Generally, components of the driver assessment system 210 are executed by a back-end system, for example, the back-end system 130 or the computing device 107 associated with the vehicle 106. As depicted, the driver assessment system 210 includes a collection module 212, a driving abilities module 214, and a driving services module 216. Generally, the driving abilities module 214 builds or trains a driving ability model 213 through, for example, machine learning and processing received driver data through the driving ability model 213 to determine a capability of the driver 122 of the vehicle 106 while the driving services module 216 builds or trains a vehicle command model 215 through, for example, machine learning and processing received capability of the driver through the trained driving ability model 213 to determine a command, which is provided to the computing device 107 for execution. In some example, execution of the determined command prevents the execution of another command(s).

In some examples, the driver assessment system 210 receives or retrieves data related to drivers or driving from the data sources 202 via the collection engine 212. In some examples, the collection engine 212 retrieves the driving data via an API. In some examples, the collection engine 212 provides an API or other service through which the various sensors and related systems provide the driving data. In some examples, the received driving data includes data collected by various sensors installed in vehicles. These vehicle sensors include, for example, steering sensors, imaging sensors (e.g., a driver monitoring camera), Light Detection and Ranging (LiDAR) sensors, radar sensors, pressure sensors, ultrasonic sensors, temperature sensors, proximity sensors, current sensors, speed sensors, steering wheel torque sensors (e.g., to determines driver input control), center console/steering wheel buttons, pedal sensors (e.g., to detect accelerator/brake pedal depressions), and so forth. In some examples, the received driving data includes data collected by smart devices associated with drivers of vehicles, for example, a smart device.

In some examples, the driving data received by the collection engine 212 includes biometric data for drivers and capability of the driver determined according to aspects to an instance of the driver assessment system. In some examples, the collected driving data includes command determined according to aspects of the system based on a determined capability of the driver. In some examples, the collection engine 212 stores the collected driving data in a data store. In some examples, the collection engine 212 provides the collected driving data to the driving ability module 214 and the driving services module 216.

In some examples, the driving ability module 214 builds or trains the driving ability model 213 with driving data received from the collection module 212. For example, the driving ability module 214 may build a specific driving ability model 213 for the driver 122 with historic driving data collected for the driver 122. For example, if the driver 122 typically sleeps for seven hours, then the driving ability model 213 account for this information so that it does not punish the driver when other drivers of the vehicle 106 are typically sleeping for eight hours. As another example, if the driver 122 exercises regularly, then drive data that reflects a recent exercise at a typical intensity level would not be considered intense for the driver 122 even though such an intensity level might be considered intense for other drivers of the vehicle 106.

Once the driving ability model 213 is trained, the driving ability module 214 processes driving data received from the computing devices 102, 104, and 107 through the driving ability model 213 to determine a current capability of the driver 122, which is provided to the driving services module 216. In some cases, the driving ability module 214 retrains the driving ability model 213 with the determined capability of the driver.

In some examples, the driving ability model 213 is employed to determine driver reaction time, driver dangerous situation recognition time, driver likelihood to drift out of lane, driver likelihood to become unconscious, time a driver's eyes are closed during driving, and so forth. In some examples, the driving ability model 213 includes weighted conditions applied to various elements of the collected driver data. For example, a driver's likelihood to become unconscious may be determined according to (0.25*(8 hours−Hours of sleep))+0.25*(caloric deficit)+0.25*(% of time driver eyes are closed)+0.25*(Heavy exercise occurred within 1 hour).

In some examples, the driving services module 216 trains the vehicle command model 215 with driving data received from the collection module 212. Once the vehicle command model 215 is trained, the driving services module 216 processes capabilities of drivers received from the driving ability module 214 through the vehicle command model 215 to determine a command for a system related to the vehicle 107 (e.g., a system executed by the computing device 107). The determined command is provided to the computing device 107 for execution. In some cases, the driving services module 216 retrains the vehicle command model 215 with the determined command.

In some examples, the determined commands include flags or Booleans that are employed to prevent, for example, handover of driving task to driver, activation of ADAS function (e.g., Adaptive Cruise Control (ACC), Automatic Emergency Braking (AEB)), or ADAS functions that require driver confirmation (e.g., lane change confirmation). In some examples, the vehicle command model 215 includes weighted conditions applied to various elements of the received capability of the driver. For example, preventing hand over of the driving task equals a likelihood that the driver will become or is unconscious >0.8 OR driver reaction time >1 second OR driver dangerous situation recognition time >1 second.

In some examples, once trained the driving ability model 213 and/or the vehicle command model 215 are provided to the computing device 107. In such examples, aspects of the driving ability module 214 and/or the driving services module 216 are executed by the computing device 107 to processes the driving data or determined capability of the driver through the respective models to determine an output (e.g., a capability of the driver or a command). In other examples, the driving ability model 213 and/or the vehicle command model 215 executed by the computing device 107 received data from the collection module 212 and train the respective models with the received data. In other examples, the driving ability module 214 and/or the driving services module 216 is executed by a back-end system that receives driver data from the computing devices 102, 104 and 107 and provides capabilities of drivers and/or command to the computing device 107 in real-time via a network, for example, communications network 110. The driver data may include, for example, recent caffeine intake, hours of sleep (rapid eye movement (REM), deep, light, and so forth), recent intense exercise, time of day, caloric or water intake over a period of time (e.g., 24 hours), and so forth.

### Example Process

FIG. 3 depicts a flowchart of an example process 300 that can be implemented by examples of the present disclosure, for example, the systems and devices depicted in FIGS. 1 and 2. The process 300 generally shows in more detail how a current capability of a driver of a vehicle is determined based on driver data received from a smart device.

For clarity of presentation, the description that follows generally describes the process 300 in the context of FIGS. 1, 2, and 4. However, it will be understood that the process 300 may be performed, for example, by other suitable system, environment, software, and hardware, or a combination of systems, environments, software, and hardware as appropriate. In some examples, various operations of the process 300 can be run in parallel, in combination, in loops, or in a different order.

At block 302, driver data that includes an indicator regarding an ability of the driver to drive the vehicle is received from a smart device associated with a driver of the vehicle. In some examples, the driver data includes key indicators of the driver's ability to recognize and react to danger. In some examples, the key indicators include biometric data, driving behavior data, and health related data. In some examples, the smart device includes an item of smart jewelry worn by the driver. In some examples, the smart device includes a mobile device coupled to a smart watch worn by the user. In some examples, the biometric data is collected by the smart watch that is provided to the mobile device. From block 302, the process 300 proceeds to block 304.

At block 304, the driver data is processed through a first model to determine a current capability of the driver. In some examples, the first model trained with previously received driver data for the driver. In some examples, the first model is retrained with the determined current capability of the driver. In some examples, the first model is retrained with the driver data. In some examples, the first model trained with previously received driver data for other drivers of the vehicle or other vehicles. In some examples, the current capability of the driver includes a measure of the ability of the driver to drive the vehicle. In some examples, the ability of the driver to drive the vehicle includes the driver's cognitive ability to perform at least one dynamic driving task. From block 304, the process 300 proceeds to block 306.

At block 306, a command to control or prevent control for the vehicle based on the current capability of the driver and a current state of the vehicle. In some examples, the command is determined by processing the current capability of the driver and the current state of the vehicle through a second model. In some examples, the second model is trained with previously received driver data for the driver, previously determined capability of the other drivers or respective vehicle states for the other vehicles. In some examples, the second model is retrained with the determined command. In some examples, the second model is retrained with the current state of the vehicle. From block 306, the process 300 proceeds to block 308.

At block 308, the command is provided to a system of the vehicle for execution. In some examples, the system includes an ADAS. In some examples, the current state of the vehicle is received from the ADAS. In some examples, the ADAS is executed by an ECU of the vehicle. In some examples, the command includes disabling a function of the vehicle or performing a minimum risk maneuver. After block 308, the process 300 ends.

In some examples, the process 300 is executed by a processor. In some examples, the processor is housed within a mobile device. In some examples, the processor is a component of an ECU of the vehicle. In some examples, the processor is housed within the smart device. In some examples, the smart device is communicably coupled to the vehicle via an infotainment system of the vehicle.

### Example System

FIG. 4 depicts an example system 400 that includes a computer or computing device 410 that can be programmed or otherwise configured to implement systems or methods of the present disclosure. For example, the computing device 410 can be programmed or otherwise configured to/as the computing devices 102, 104, 107, or 132 described above with reference to FIGS. 1 and 2.

In the depicted example, the computer or computing device 410 includes an electronic processor (also “processor” and “computer processor” herein) 412, which is optionally a single core, a multi core processor, or a plurality of processors for parallel processing. The depicted example also includes memory 417 (e.g., random-access memory, read-only memory, flash memory), electronic storage unit 414 (e.g., hard disk or flash), communication interface 415 (e.g., a network adapter or modem) for communicating with one or more other systems, and peripheral devices 416, for example, cache, other memory, data storage, microphones, speakers, and the like. In some examples, the memory 417, storage unit 414, communication interface 415 and peripheral devices 416 are in communication with the electronic processor 412 through a communication bus (shown as solid lines), for example, a motherboard. In some examples, the bus of the computing device 410 includes multiple buses. In some examples, the computing device 410 includes more or fewer components than those illustrated in FIG. 4 and performs functions other than those described herein.

In some examples, the memory 417 and storage unit 414 include one or more physical apparatuses used to store data or programs on a temporary or permanent basis. In some examples, the memory 417 is volatile memory and requires power to maintain stored information. In some examples, the storage unit 414 is non-volatile memory and retains stored information when the computer is not powered. In further examples, memory 417 or storage unit 414 is a combination of devices for example, those disclosed herein. In some examples, memory 417 or storage unit 414 is distributed across multiple machines for example, a network-based memory or memory in multiple machines performing the operations of the computing device 410.

In some cases, the storage unit 414 is a data storage unit or data store for storing data. In some instances, the storage unit 414 store files, for example, drivers, libraries, and saved programs. In some examples, the storage unit 414 stores user data (e.g., user preferences and user programs). In some examples, the computing device 410 includes one or more additional data storage units that are external, for example, located on a remote server that is in communication through an intranet or the internet.

In some examples, methods as described herein are implemented by way of machine or computer executable code stored on an electronic storage location of the computing device 410, for example, on the memory 417 or the storage unit 414. In some examples, the electronic processor 412 is configured to execute the code. In some examples, the machine executable or machine-readable code is provided in the form of software. In some examples, during use, the code is executed by the electronic processor 412. In some cases, the code is retrieved from the storage unit 414 and stored on the memory 417 for ready access by the electronic processor 412. In some situations, the storage unit 414 is precluded, and machine-executable instructions are stored on the memory 417.

In some cases, the computing device 410 includes or is in communication with one or more output devices 420. In some cases, the output device 420 includes a display to send visual information to a user. In some cases, the output device 420 is a touch sensitive display that combines a display with a touch sensitive element that is operable to sense touch inputs as and functions as both the output device 420 and the input device 430. In still further cases, the output device 420 is a combination of devices for example, those disclosed herein. In some cases, the output device 420 displays a user interface (UI) 425 generated by the computing device (for example, software executed by the computing device 410).

In some cases, the computing device 410 includes or is in communication with one or more input devices 430 that are configured to receive information from a user. Suitable input devices include a keyboard, a cursor-control device, a touchscreen, a microphone, and a camera.

In some cases, the computing device 410 includes an operating system configured to perform executable instructions. The operating system is, for example, software, including programs and data that manages the device's hardware and provides services for execution of applications.

### Machine Learning

In some examples, machine learning algorithms are employed to build a model to determine a current capability of a driver. In some examples, machine learning algorithms are employed to build a model to determine a command for a vehicle system. Examples of machine learning algorithms may include a support vector machine (SVM), a naïve Bayes classification, a random forest, a neural network, deep learning, or other supervised learning algorithm or unsupervised learning algorithm for classification and regression. The machine learning algorithms may be trained using one or more training datasets. For example, previously received driving data and/or previously determined capabilities of drivers and vehicle system commands may be employed to train various algorithms. Moreover, as described above, these algorithms can be continuously trained/retrained using real-time user data as it is received. In some examples, the machine learning algorithm employs regression modeling where relationships between variables are determined and weighted. In some examples, the machine learning algorithm employ regression modeling, wherein relationships between predictor variables and dependent variables are determined and weighted.

In the foregoing specification, specific examples have been described. However, various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a,” “has . . . a,” “includes . . . a,” or “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially,” “essentially,” “approximately,” “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way but may also be configured in ways that are not listed. The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A system for determining a current capability of a driver of a vehicle, comprising:
a processor configured to
receive, from a smart device associated with a driver of the vehicle, driver data that includes an indicator regarding an ability of the driver to drive the vehicle;
process the driver data through a first model to determine a current capability of the driver, the first model trained with previously received driver data for the driver;
determine, based on the current capability of the driver and a current state of the vehicle, a command to control or prevent control of the vehicle; and
provide the command to a system of the vehicle for execution.

2. The system of claim 1, wherein the first model is retrained with the determined current capability of the driver.

3. The system of claim 1, wherein the first model is retrained with the driver data.

4. The system of claim 1, wherein the first model trained with previously received driver data for other drivers of the vehicle or other vehicles.

5. The system of claim 1, wherein the command is determined by processing the current capability of the driver and the current state of the vehicle through a second model.

6. The system of claim 5, wherein the second model is trained with previously received driver data for the driver, previously determined capability of the other drivers, or respective vehicle states for the other vehicles.

7. The system of claim 5, wherein the second model is retrained with the determined command or the current state of the vehicle.

8. The system of claim 1, wherein the current capability of the driver includes a measure of the ability of the driver to drive the vehicle.

9. The system of claim 8, wherein the ability of the driver to drive the vehicle includes the driver's cognitive ability to perform at least one dynamic driving task.

10. The system of claim 1, wherein the driver data includes key indicators of the driver's ability to recognize and react to danger, wherein the key indicators include biometric data, driving behavior data, and health related data.

11. The system of claim 1, wherein the system comprises an Advanced Driver Assistance System (ADAS), and wherein the current state of the vehicle is received from the ADAS.

12. The system of claim 11, wherein the ADAS is executed by an electronic control unit (ECU) of the vehicle.

13. The system of claim 1, wherein the smart device comprises an item of smart jewelry worn by the driver.

14. The system of claim 1, wherein the smart device comprises a mobile device coupled to a smart watch worn by the user, and wherein the biometric data is collected by the smart watch that is provided to the mobile device.

15. The system of claim 1, wherein the command includes disabling a function of the vehicle or performing a minimum risk maneuver.

16. The system of claim 1, wherein the processor is housed within a mobile device.

17. The system of claim 1, wherein the processor is a component of an electronic control unit (ECU) of the vehicle.

18. The system of claim 1, wherein the processor is housed within the smart device that is communicably coupled to the vehicle via an infotainment system of the vehicle.

19. A method for determining a current capability of a driver of a vehicle, the method comprising:
receiving, from a smart device associated with a driver of the vehicle, driver data that includes an indicator regarding an ability of the driver to drive the vehicle;
processing the driver data through a first model to determine a current capability of the driver, the first model trained with previously received driver data for the driver;
determining, based on the current capability of the driver and a current state of the vehicle, a command to control or prevent control of the vehicle; and
providing the command to a system of the vehicle for execution.

20. A non-transitory computer-readable medium including instructions executable by an electronic processor to perform a set of functions, the set of functions comprising:
receiving, from a smart device associated with a driver of the vehicle, driver data that includes an indicator regarding an ability of the driver to drive the vehicle;
processing the driver data through a first model to determine a current capability of the driver, the first model trained with previously received driver data for the driver;
determining, based on the current capability of the driver and a current state of the vehicle, a command to control or prevent control of the vehicle; and
providing the command to a system of the vehicle for execution.
